(19) United States
(12) Patent Application Publication
(10) Pub. No.: US 2026/0243684 A1
(43) Pub. Date: Aug. 20, 2026

(54) PORTABLE MULTI-CHANNEL FLUORESCENCE DETECTOR

(71) Applicants: UIF (University Industry Foundation), Yonsei University, Seoul (KR); Korea Research Institute of Bioscience and Biotechnology, Daejeon (KR)

(72) Inventors: Seungjoo HAAM, Seoul (KR); Seung Jae Oh, Gwangmyeong-si (KR); Eun Kyung Lim, Daejeon (KR); Hyoung Hwa Jeong, Seoul (KR); Sojeong Lee, Seoul (KR); Inhee Maeng, Goyang-si (KR)

(21) Appl. No.: 19/539,733

(22) Filed: Feb. 13, 2026

(30) Foreign Application Priority Data
Feb. 18, 2025 (KR) 10-2025-0021168

(51) Int. Cl. G01N 21/64; G01N 33/58

(52) U.S. Cl. CPC G01N 21/6458; G01N 21/6428; G01N 21/6452; G01N 21/6456; G01N 21/6486; G01N 33/582

(57) ABSTRACT

A portable multi-channel fluorescence detector that can be used to enable the simultaneous diagnosis of two or more types of target viruses and provide precise field diagnosis for large-scale population samples.

### CROSS-REFERENCE TO RELATED APPLICATION

This application claims priority to and the benefit of Korean Patent Application No. 10-2025-0021168, filed on February 18, 2025, the disclosure of which is incorporated herein by reference in its entirety.

### BACKGROUND

### 1. Field of the Invention

The present invention relates to a portable multi-channel fluorescence detector. In addition, the present invention relates to a method of detecting two or more types of viruses using a portable multi-channel fluorescence detector.

### 2. Discussion of Related Art

Respiratory viruses have the ability to survive in the air for long periods and spread rapidly. These viruses have a high likelihood of reemerging as variants, and their symptoms are highly similar across diseases, making it difficult to accurately trace the exact timing of infection. Therefore, precise diagnostic tools are needed to prevent the misuse of pharmaceutical treatments even after infection.

qPCR still serves as the golden standard due to its high sensitivity and specificity. However, the complexity of sample preparation, the need for multiple amplification steps, and the use of enzymes pose barriers to its application to large-scale field testing.

In contrast, immunoassays can identify a wide range of targets, including various biomarkers, without the need for nucleic acid extraction or complex primer design. In addition, they have the advantage of reducing the risk of cross-contamination due to pipetting and accelerating the delivery of results due to fewer processing steps.

herefore, integration of immunoassays with portable fluorescence signal analyzers can expand the possibilities of precise field diagnosis for large-scale population samples.

### SUMMARY OF THE INVENTION

In order to early detect disease and prevent the spread of infectious diseases, low-concentration biomarker detection technology needs to be involved. To establish such a system, it is essential to develop an ultra-sensitive detection technology that can separate and detect only biomarkers from non-specific proteins in a sample.

Therefore, the present invention is directed to providing a novel nanoprobe for target virus detection.

The present invention is also directed to providing a portable multi-channel fluorescence detector that detects a target virus by applying the novel nanoprobe.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a schematic diagram showing a method of detecting viruses using a portable multi-channel fluorescence detector;

FIGS. 2A, 2B and 2C show a portable multi-channel fluorescence detector according to an example of the present invention;

FIG. 3 shows a fluorescence cartridge according to an example of the present invention;

FIG. 4 shows a program flow chart using a portable multi-channel fluorescence detector according to an example of the present invention;

FIG. 5 shows the results of comparing the performance of an existing multi-reader and a portable multi-channel fluorescence detector after performing an immunoassay; and

FIG. 6 shows the results of treating artificial saliva and artificial nasal fluid with polymeric nanoprobes and analyzing the signals using a portable multi-channel fluorescence detector.

### DETAILED DESCRIPTION OF EXEMPLARY EMBODIMENTS

The present invention relates to a portable multi-channel fluorescence detector.

Hereinafter, a portable multi-channel fluorescence detector 100 of the present invention will be described in detail with reference to the drawings.

The multi-channel fluorescence detector 100 of the present invention includes: a receiving unit 120 in which a sample is loaded; a fluorescence cartridge 110 that detects a fluorescence wavelength from the sample; a main body 130 in which two or more fluorescence cartridges are loaded; and a display unit 140 that is exposed to the outside of the main body and displays detected fluorescence results.

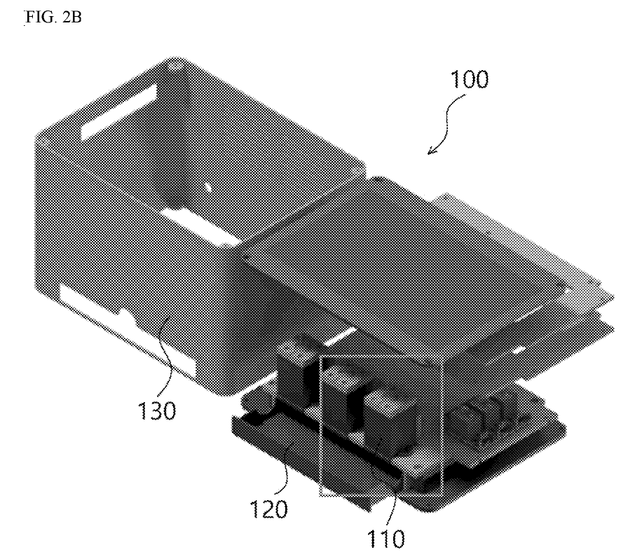

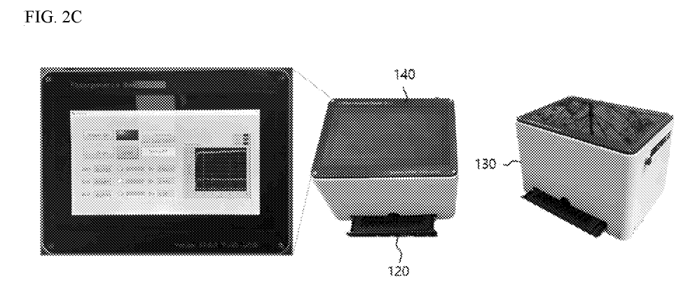

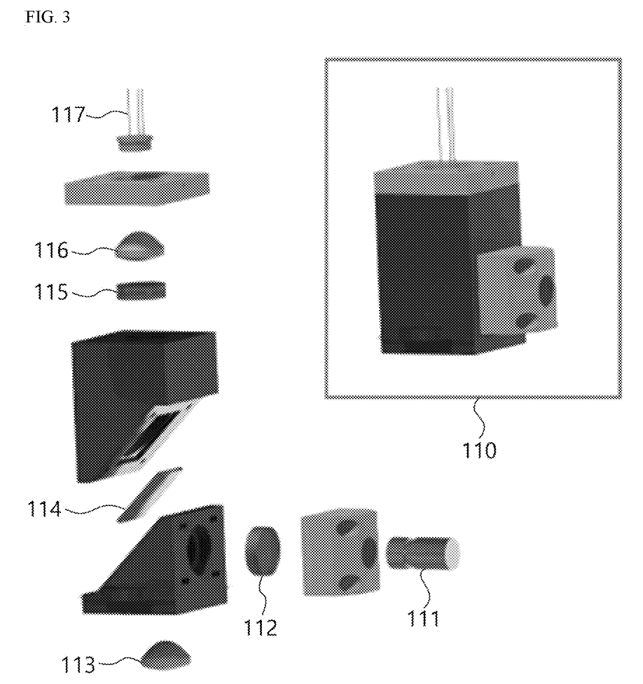

In the present invention, FIGS. 2A, 2B and 2C show a portable multi-channel fluorescence detector according to an example of the present invention, and FIG. 3 shows a fluorescence cartridge.

In the present invention, a sample is introduced into the receiving unit (plate inlet) 120. The receiving unit 120 is coupled to the main body 130, and the sample in the receiving unit may be loaded on the fluorescence cartridge 110.

In one embodiment, the sample may include a fluorescent reagent, and the fluorescent reagent may be a fluorescent molecule. In an embodiment of the present invention, an antibody-conjugated nanoprobe loaded with a fluorescent molecule may be used, and a signal in a concentration range of 100 to 500 nM may be detected based on the antibody-conjugated nanoprobe.

In the present invention, the fluorescence cartridge 110 is located inside the main body 130, a sample of the receiving unit is loaded therein, and the fluorescence wavelength may be detected from the sample. In the present invention, a variety of laser combinations to be analyzed may be secured using the fluorescence cartridge.

In the present invention, the fluorescence cartridge 110 may include a first space and a second space partitioned by the loaded sample as a boundary. Light irradiated by a light source may pass through the first space, pass through the sample in the receiving unit, and move through the second space to reach a detection unit.

In one embodiment, the first space is an excitation space, and may include: a light source 111; a first optical filter 112 that passes light of a predetermined wavelength among the light irradiated from the light source 111; and a first condensing lens 113 that condenses the light toward the sample along an optical path.

In one embodiment, the light source (laser) 111 may be an LED light source. When light is irradiated from the light source toward the sample, the light collides with particles of the substance of interest in the sample, and the light excites electrons of specific molecules, causing fluorescence.

In one embodiment, the first optical filter 112 may be an excitation filter and may be disposed on an optical path connecting the light source and the sample. The first optical filter may pass light of a predetermined wavelength among the light irradiated from the light source.

In one embodiment, the first condensing lens 113 may condense the light passed through the first optical filter toward the sample.

In one embodiment, a dichroic filter 114 may be further included between the first space and the second space. The dichroic filter may assist the functions of a first optical filter and a second optical filter to transmit an emission beam and reflect an excitation beam.

In the present invention, the second space is an emission measurement space, and may include: a second optical filter 115 that passes light of a predetermined wavelength among the light passing through the receiving unit; a second condensing lens 116 that condenses the light toward a detection unit; and the detection unit 117 along the optical path.

In one embodiment, the second optical filter 115 is an emission filter and is positioned on an optical path connecting the receiving unit and the detection unit. The second optical filter may pass light of a predetermined wavelength among the light passing through the sample.

In one embodiment, the second condensing lens 116 may condense the light passed through the second optical filter toward the detection unit.

In one embodiment, the detection unit (photodiode) 117 may be disposed within the second space to detect fluorescence from the sample and output an electrical signal corresponding to the intensity of the fluorescence.

The detection unit 117 may include a photodetector or CCD.

In one embodiment, the first optical filter 112 and the second optical filter 115 may vary depending on the type of fluorescent reagent. For example, when DiO is used as the fluorescent reagent, the first and second optical filters may be provided to pass light of 470 to 485 nm. In addition, when DiI is used as the fluorescent reagent, the first and second optical filters may be provided to pass light of 520 to 535 nm. In addition, when DiD is used as the fluorescent reagent, the first and second optical filters may be provided to pass light of 620 to 635 nm.

In the present invention, the fluorescence cartridge 110 may be configured such that the first space and the second space are orthogonal to the sample.

In the present invention, the number of fluorescence cartridges 110 loaded in the main body may be two or more, specifically, three to five. The present invention uses a plurality of fluorescence cartridges, and thus each fluorescence cartridge is capable of detecting one target virus. The use of such fluorescence cartridges increases portability and eliminates the need for optical filter replacement.

In the present invention, the fluorescence cartridge 110 may have dimensions including a width ranging from 20 to 80 mm, a length ranging from 20 to 80 mm, and a height ranging from 20 to 50 mm, or a width ranging from 40 to 60 mm, a length ranging from 40 to 60 mm, and a height ranging from 25 to 40 mm. It is easy to carry within the above dimensions. In addition, since one virus is targeted per fluorescence cartridge, when the fluorescence cartridge is manufactured in advance according to the target virus, it has the advantage of enabling detection of various types of viruses by simply exchanging fluorescence cartridges by on-site attachment and detachment.

In the present invention, due to its compact, portable structure, a housing forming the exterior of the fluorescence cartridge 110 may be made of a metal material in order to easily dissipate heat generated during the light detection process to the outside.

In the present invention, the main body 130 is a skeleton of the portable multi-channel fluorescence detector, with the fluorescence cartridge 110 loaded therein and the receiving unit 120 loaded/unloaded therein.

In one embodiment, two or more, specifically three to five, fluorescence cartridges 110 are loaded in the main body 130, enabling detection of various types of target viruses.

In one embodiment, the main body 130 may have dimensions including a width ranging from 10 to 30 cm, a length ranging from 10 to 30 cm, and a height ranging from 10 to 30 cm, or a width ranging from 15 to 25 cm, a length ranging from 10 to 20 cm, and a height ranging from 10 to 20 cm, and is easy to carry, allowing for easy on-site detection of target viruses.

In one embodiment, the housing forming the exterior of the main body 130 may be made of metal.

The portable multi-channel fluorescence detector according to the present invention includes a display unit 140 that is exposed to the outside of the main body and displays the detected fluorescence results. The display unit may be a touch screen. The touch screen may use Windows-based software.

The portable multi-channel fluorescence detector according to the present invention may further include a communication interface (not shown) for communicating with the outside.

In one embodiment, the communication interface may be LAN, Wifi, HDMI, and the like.

The portable multi-channel fluorescence detector according to the present invention may further include a charging terminal or USB port as needed.

A detection system using the portable multi-channel fluorescence detector according to the present invention includes: a detection unit that detects the insertion of a sample; an optical unit for irradiating light toward the sample and detecting light passing through the sample; a display unit for displaying the detected fluorescence results; and a control unit for operating the optical unit based on the detection results of the detection unit and outputting the detection results to the outside.

In the present invention, the detection unit may detect the insertion of a sample using various types of sensors used in the art. Next, when the insertion of the sample is detected, the insertion detection is displayed on the display unit so that the user can visually confirm the insertion of the sample.

In the present invention, the optical unit can irradiate light toward the sample and detect light passing through the sample. Such light irradiation and detection may be performed within the fluorescence cartridge.

In one embodiment, the display unit can operate the optical unit based on the detection result of sample insertion, and can output the detection result to the outside.

In one embodiment, the control unit may function to externally display the detection result when the detected fluorescence sensitivity is greater than or equal to a reference fluorescence sensitivity. For example, the control unit can determine that the substance is present when the concentration of the target virus is above a predetermined value.

In one embodiment, when sample insertion is detected by the sensor, the control unit can automatically operate the optical unit without separate user input and output the detection result to the outside. Therefore, simply by inserting a sample into the receiving unit, it is possible to easily check whether the target virus in the sample reacts or not without a separate input operation.

In addition, the control unit may control the optical detection order of the sample. For example, when analyzing three samples using three fluorescence cartridges, the analysis may be controlled to proceed sequentially in the order of a first fluorescence cartridge, a second fluorescence cartridge, and a third fluorescence cartridge. This sequential application may prevent optical interference during analysis.

In addition, the present invention relates to a method of detecting two or more types of viruses using the above-described portable multi-channel fluorescence detector.

The method of detecting viruses according to the present invention includes: (S1) loading a sample in the portable multi-channel fluorescence detector; and (S2) detecting a fluorescence signal of the sample to determine whether a virus is present.

Step (S1) of the present invention is a step of loading a sample in the portable multi-channel fluorescence detector, wherein the sample may be prepared by treating the sample with antibody-conjugated nanoprobes and antibody-conjugated magnetic nanoparticles and allowing them to react.

In the present invention, the nanoprobe includes: an amphiphilic polymeric nanoparticle having a bilayer membrane structure; a type of hydrophobic fluorescent molecule loaded into a hydrophobic portion of the amphiphilic polymer membrane; and an antibody attached to the surface of the amphiphilic polymeric nanoparticle.

The nanoprobe according to the present invention may maintain a self-quenching state without a quencher by inducing self-quenching through energy release and absorption between fluorescent molecules loaded into a narrow hydrophobic portion.

In the present invention, the amphiphilic polymer is a polymer that exhibits amphiphilic properties by combining a hydrophilic polymer and a hydrophobic polymer.

In one embodiment, the amphiphilic polymer may have a hydrophobic fraction of 0.28 or less. Under the above conditions, nanoparticles with a bilayer membrane structure are stably formed and may load more fluorescent molecules.

In one embodiment, the amphiphilic polymeric nanoparticle may have a particle structure in which the hydrophilic portion of the amphiphilic polymer is aggregated on the inside and the hydrophobic portion is located on the outside.

The hydrophilic polymer may be one or more selected from the group consisting of polyethylene glycol (PEG), polyacrylic acid (PAA), polyethylene oxide (PEO), and polyvinyl acetate (PVA).

The hydrophobic polymer may be one or more selected from the group consisting of polylactic acid (PLA) and poly(L-leucine).

The amphiphilic polymer may be PEG-b-PLA or PEG-b-pLeu.

In one embodiment, the amphiphilic polymeric nanoparticle may have a particle size of 80 to 120 nm. When the particle size is less than 80 nm, a self-quenching phenomenon is minimal, making signal analysis difficult, and when the particle size exceeds 120 nm, there is a problem that a background signal increases and the concentration of fluorescent molecules becomes too high, lowering the stability of the particles.

In the present invention, the fluorescent molecule may be loaded into the hydrophobic portion of the amphiphilic polymer membrane.

In one embodiment, the fluorescent molecule may be hydrophobic, allowing it to be loaded into the hydrophobic portion of the amphiphilic polymeric nanoparticle. In the case of a hydrophilic fluorescent molecule, stable loading is difficult, so it is desirable to have hydrophobicity.

In one embodiment, the fluorescent molecule may have an orbital in which the energy difference between the LUMO and HOMO is 6.45 eV or less. The smaller the energy difference between the LUMO and HOMO, the easier it is to absorb energy emitted by surrounding molecules, and thus the fluorescent molecule of the present invention may easily induce self-quenching.

In one embodiment, the type of fluorescent molecule is not particularly limited, and may be one or more selected from the group consisting of 3,3-dioctadecyloxacarbocyanine perchlorate (Dio; Dioc), 1,1-dioctadecyl-3,3,3,3-tetramethylindocarbocyanine perchlorate (DiI), 4,4-diisothiocyanatostilbene-2,2-disulfonic acid disodium salt (DID, DIDS), 4-(4-(dihexadecylamino)styryl)-N-methylpyridinium iodide (DiA; 4-Di-16-ASP), and 1,1-dioctadecyl-3,3,3,3-tetramethylindotricarbocyanine iodide (DiR).

In one embodiment, 50 or more or 60 to 80 fluorescent molecules may be loaded per amphiphilic polymeric nanoparticle. When 50 or more fluorescent molecules are loaded, self-quenching may occur.

In the present invention, an antibody is attached to the surface of the amphiphilic polymeric nanoparticle, and the antibody may bind to the target virus.

In one embodiment, each amphiphilic polymeric nanoparticle may have a type of antibody attached thereto.

In one embodiment, different fluorescent molecules may be attached depending on the type of antibody. Specifically, when three types of antibodies are used, three types of fluorescent molecules may be used. A first antibody and a first fluorescent molecule may be loaded and attached to one amphiphilic polymeric nanoparticle, a second antibody and a second fluorescent molecule may be loaded and attached to another amphiphilic polymeric nanoparticle, and a third antibody and a third fluorescent molecule may be loaded and attached to still another amphiphilic polymeric nanoparticle. This enables the detection of one type of target virus per one type of nanoprobe.

In the present invention, the magnetic nanoparticles may be metal oxides, preferably iron oxides. Antibodies may be conjugated to the magnetic nanoparticles.

In one embodiment, the magnetic nanoparticle may have a particle size of 200 nm or less. This particle size has a large surface area and thus excellent separation efficiency may be achieved.

In the present invention, the nanoprobe and the antibody-conjugated magnetic nanoparticles may bind to the target virus to form an immune complex. Specifically, the immune complex may have a structure of nanoprobe-target virus-magnetic nanoparticle. That is, in the present invention, the sample may include an immune complex.

In the present invention, the nanoprobe and the magnetic nanoparticles may be allowed to react at a volume ratio of 1:1. When the ratio of the magnetic nanoparticles exceeds 1, it may adversely affect signal detection by absorbing fluorescence; therefore, it is preferable to use a volume ratio of 1:1.

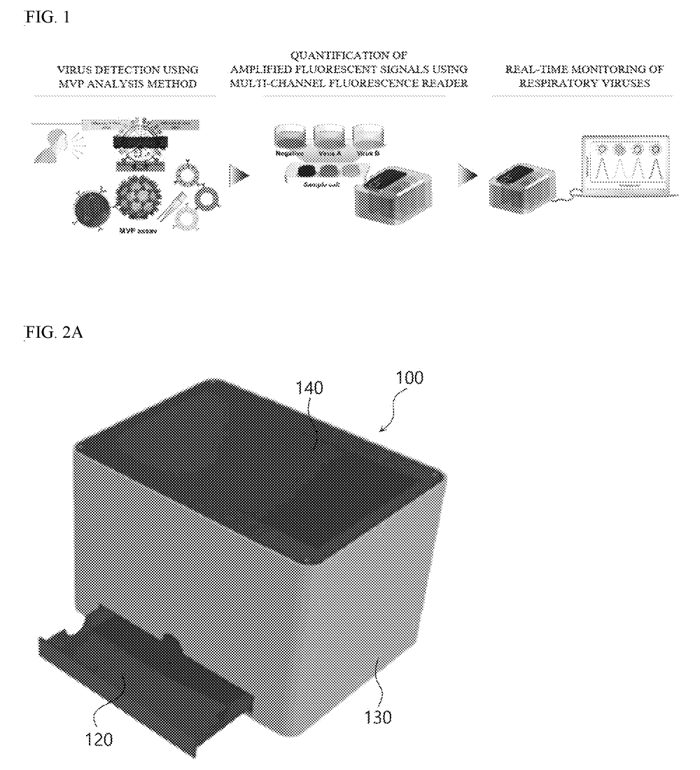

The present invention may detect a target virus using the portable multi-channel fluorescence detector (FIG. 1).

In the present invention, when a sample is loaded in the receiving unit of the fluorescence cartridge and when light is irradiated according to a program, the fluorescent detector may detect the fluorescence signal to determine whether a virus is present.

In one embodiment, the detection of the fluorescence signal may be performed by detecting a fluorescence signal from one fluorescence cartridge and then detecting a fluorescence signal from the next fluorescence cartridge.

The portable multi-channel fluorescence detector according to the present invention may detect a plurality of viruses.

Hereinafter, the present invention will be described in detail through examples. The following examples merely illustrate the present invention and are not intended to limit the scope of the present invention.

### Example 1. Performance comparison with existing multi-reader

The performance of a field-portable multi-channel fluorescence detector according to the present invention was compared with that of an existing multi-reader (product name: SpectraMax^{®} i3x Multi-Mode Microplate Reader, manufacturer: Molecular Devices).

FIG. 5A shows an analysis method using the portable multi-channel fluorescence detector according to the present invention.

### 1 Manufacture of antibody-conjugated nanoprobes

An amphiphilic polymer (PEG-b-PLA) with a hydrophobic fraction of 0.22 was dissolved in DMSO to prepare an amphiphilic polymer solution (concentration: 10 mg/ml). In addition, three types of fluorescent molecules were each dissolved in DMSO to prepare a fluorescent molecule solution (concentration: 1 mg/ml).

At this time, 3,3-dioctadecyloxacarbocyanine perchlorate (Dio; Dioc), 1,1-dioctadecyl-3,3,3,3-tetramethylindocarbocyanine perchlorate (DiI), and 4,4-diisothiocyanatostilbene-2,2-disulfonic acid disodium salt (DID, DIDS) were used as the fluorescent molecules.

1 ml of the fluorescent molecule solution was treated with 1 ml of the amphiphilic polymer solution and mixed with 9.9 ml of distilled water. After inducing self-assembly at room temperature for 12 hours or more with strong vortexing, the mixture was dialyzed for 2 days using a 100 kDa membrane.

Next, the surfaces of three types of nanoprobes were modified with antibodies to prepare antibody-conjugated nanoprobes.

The antibodies are antibodies against three type of target viruses (IAV, IBA, and Corona).

### 2 Manufacture of antibody-conjugated magnetic nanoparticles

The surface of iron oxide magnetic nanoparticles with a particle size of 200 nm was modified with antibodies to prepare antibody-conjugated magnetic nanoparticles. When the antibody was attached, a silane-PEG-COOH linker was used, and a PEG molecular weight was 2 kDa.

### 3 Sample preparation

The antibody-conjugated nanoprobe (concentration: 0.5 to 2 nM) prepared in (1) and the antibody-conjugated magnetic nanoparticles (concentration: 0.5 to 2 nM) prepared in (2) were added to a mixed solution of three types of viruses (IAV, IBA, Corona) at a volume ratio of 1:1 and allowed to react.

At this time, the virus concentration was fixed at 10³ TCID₅₀/mL for each sample, and three types of samples were prepared. The method of preparing the sample is as follows.

First, a magnet was placed on the mixed solution to separate the antibody-conjugated nanoprobe-virus-antibody-conjugated magnetic nanoparticles (immune complex), and then washed three times with DIW. Next, the immune complex was treated with a lysis solution (TX-100 or Tween 20, 0.05-10 wt%) and allowed to react at 37 °C for 10 minutes. Finally, a magnet was applied to obtain only the fluorescent particles in the supernatant, which were used for detection.

### 4 Virus detection

The three types of samples manufactured in (3) were each loaded in the receiving units of three fluorescence cartridges, and then loaded in the main body of a portable multi-channel fluorescent detector.

Through this, a fluorescence analysis was performed.

The analysis method is as follows.

A 96-well plate was treated with 90 ul of nanoprobe solution at each concentration (0, 100, 200, 300, 400, and 500 pM) and 10 ul of lysis solution and allowed to react at 37 °C for 10 minutes. The same conditions were applied to three wells, and the results obtained by repeating the measurements three times were expressed as a graph. Measurements were performed on a multi-reader and also on a field-portable fluorescence reader.

Next, a regression line was drawn and an R² value was derived.

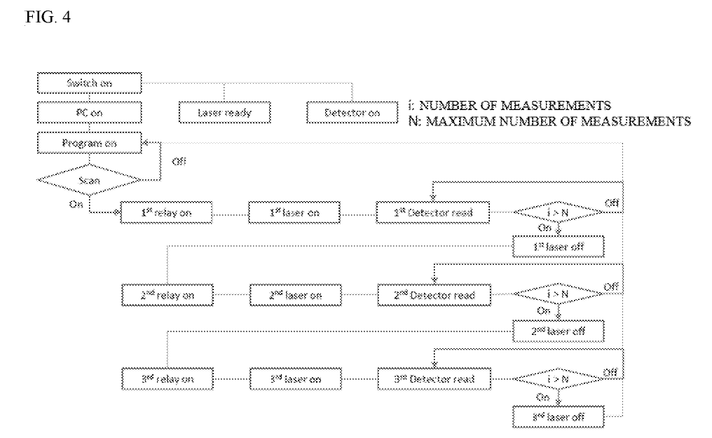

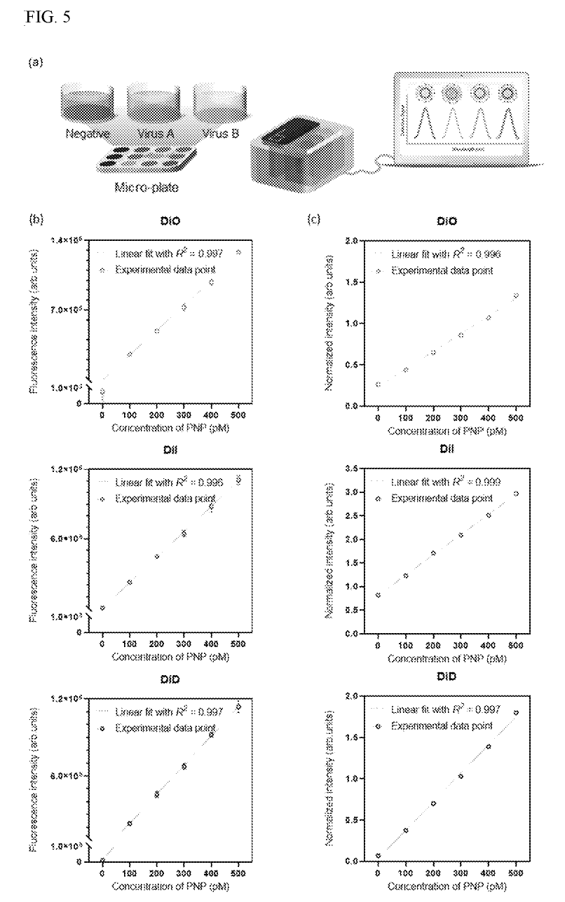

FIG. 4 shows a program flow chart of a fluorescence detector using three fluorescence cartridges, wherein a first fluorescence cartridge, a second fluorescence cartridge, and a third fluorescence cartridge may be sequentially analyzed to minimize optical interference.

### 5 Results

FIG. 5 shows the results of comparing the performance of an existing multi-reader and a 3-channel fluorescence reader after performing an immunoassay.

FIG. 5B shows the results measured with an existing multi-reader, and FIG. 5C shows the results measured with a fluorescent reader according to the present invention.

As shown in FIG. 5, a linear regression line was shown, confirming the ability to quantitatively evaluate at a very good level. In addition, it can be confirmed that similar analysis results are shown when compared to the existing multi-reader. Therefore, it can be confirmed that it is applicable as a portable detection device that can replace expensive equipment.

### Example 2. Efficacy evaluation in various solutions

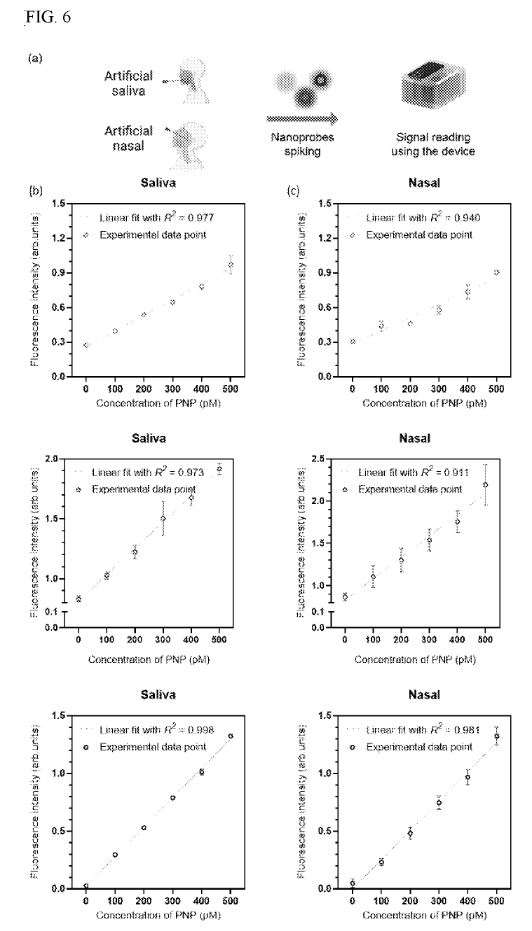

Artificial saliva and artificial nasal fluid were with the polymeric nanoprobes, and the signals were analyzed using a fluorescence reader (FIG. 6A).

Specifically, polymeric nanoprobes at a concentration of 10 nM were diluted in artificial nasal fluid (or artificial saliva) to obtain concentrations of 100 and 500 pM, respectively.

At this time, when preparing a 100 pM concentration solution, a total of 1 ml of solution was prepared with 10 ul of 10 nM concentration polymeric nanoprobes and 990 ul of artificial nasal fluid (or artificial saliva), and finally a 100 pM solution diluted 100-fold from 10 nM was prepared.

In the present invention, FIG. 6 shows the results of an analytical efficacy evaluation.

Specifically, FIG. 6B shows the results of treating artificial saliva with the polymeric nanoprobes, and FIG. 6C shows the results of treating artificial nasal fluid with the polymeric nanoprobes. As shown in the drawing, it can be confirmed that the fluorescent reader according to the present invention enables quantitative evaluation of fluorescent signals in various solutions.

A virus detection method according to the present invention exhibits a magnetic separation effect, a fluorescent self-quenching effect, and a fluorescence signal amplification effect in the presence of a target using magnetic nanoparticles and nanoprobes. This can improve sensitivity by lowering background signals and amplifying target signals. In addition, the detection method of the present invention is a diagnostic system based on an immune sandwich technique configured without a quencher or enzyme, and can simultaneously diagnose two or more types of target viruses.

In addition, the present invention can use a portable multi-channel fluorescence detector to provide precise field diagnosis for large-scale population samples. In particular, three types of fluorescence signals can be simultaneously detected within 15 seconds through 30 laser irradiations for 3 seconds.

What is claimed is:

1. A portable multi-channel fluorescence detector comprising:
a receiving unit in which a sample is loaded;
a fluorescence cartridge that detects a fluorescence wavelength from the sample;
a main body in which two or more fluorescence cartridges are loaded; and
a display unit that is exposed to the outside of the main body and displays detected fluorescence results.

2. The portable multi-channel fluorescence detector of claim 1, wherein the fluorescence cartridge includes a first space and a second space,
the first space includes: a light source; a first optical filter that passes light of a predetermined wavelength among the light irradiated from the light source; and a first condensing lens that condenses the light toward the receiving unit along an optical path, and
the second space includes: a second optical filter that passes light of a predetermined wavelength among the light passing through the receiving unit; a second condensing lens that condenses the light toward a detection unit side; and the detection unit along an optical path.

3. The portable multi-channel fluorescence detector of claim 1, wherein the number of fluorescence cartridges loaded in the main body is 3 to 5.

4. The portable multi-channel fluorescence detector of claim 1, wherein one fluorescence cartridge detects one type of virus.

5. The portable multi-channel fluorescence detector of claim 1, wherein the fluorescence cartridge has dimensions including a width ranging from 20 to 80 mm, a length ranging from 20 to 80 mm, and a height ranging from 20 to 50 mm.

6. The portable multi-channel fluorescence detector of claim 1, wherein the main body has dimensions including a width ranging from 10 to 30 cm, a length ranging from 10 to 30 cm, and a height ranging from 10 to 30 cm.

7. The portable multi-channel fluorescence detector of claim 1, further comprising a communication interface that is exposed to the outside of the main body and communicates with the outside.

8. A method of detecting viruses, comprising:
loading a sample prepared by treating the sample with an antibody-conjugated nanoprobe and antibody-conjugated magnetic nanoparticles and reacting the sample in the portable multi-channel fluorescence detector of claim 1; and
detecting a fluorescence signal of the sample to determine whether a virus is present,
wherein two or more samples with different antibodies are respectively loaded in two or more fluorescence cartridges.

9. The method of claim 8, wherein the antibody-conjugated nanoprobe includes:
an amphiphilic polymeric nanoparticle having a bilayer membrane structure;
a fluorescent molecule loaded into a hydrophobic portion of the amphiphilic polymeric nanoparticle; and
an antibody attached to a surface of the amphiphilic polymeric nanoparticle, and
maintains a self-quenching state without a quencher.

10. The method of claim 9, wherein the amphiphilic polymer includes:
one or more hydrophilic polymers selected from the group consisting of polyethylene glycol (PEG), polyacrylic acid (PAA), polyethylene oxide (PEO), and polyvinyl acetate (PVA); and
one or more hydrophobic polymers selected from the group consisting of polylactic acid (PLA) and poly(L-leucine).

11. The method of claim 9, wherein the fluorescent molecule is a hydrophobic fluorescent molecule having an orbital of 6.45 Ev or less.

12. The method of claim 9, wherein the amphiphilic polymeric nanoparticle has a particle size of 80 to 120 nm.

13. The method of claim 9, wherein the antibody-conjugated nanoprobe is loaded with different fluorescent molecules depending on the type of antibody.

14. The method of claim 8, wherein the detecting of the fluorescence signal includes detecting a fluorescence signal from one fluorescence cartridge and then detecting a fluorescence signal from the next fluorescence cartridge.
